(19) United States
(12) Patent Application Publication
(10) Pub. No.: US 2012/0283565 A1
(43) Pub. Date: Nov. 8, 2012

(54) APPARATUS AND METHOD FOR GUIDED CHRONIC TOTAL OCCLUSION PENETRATION

(75) Inventor: Jacob RICHTER, Arsuf (IL)

(73) Assignee: OSCILLON LTD., Tel Aviv (IL)

(21) Appl. No.: 13/553,659

(22) Filed: Jul. 19, 2012

Related U.S. Application Data
(63) Continuation of application No. 12/126,665, filed on May 23, 2008.
(60) Provisional application No. 60/939,766, filed on May 23, 2007.

(51) Int. Cl. A61B 8/12; A61F 2/84; A61M 25/10; A61B 5/02; A61B 17/22

(52) U.S. Cl. 600/439; 600/481

(57) ABSTRACT

An apparatus and method for guided penetration of a chronic total occlusion in a blood vessel are disclosed. The invention is directed to an apparatus that facilitates accurate placement of a drilling tip within a body lumen using ultrasound-based detection to determine the position of the intravascular catheter relative to the vessel occlusion and vessel walls.

This application claims benefit of priority under 35 U.S.C. §120 to U.S. Provisional Application Ser. No. 60/939,766 filed May 23, 2007, entitled “Apparatus and method for ultrasound imaging guided chronic total occlusion penetration.”

### FIELD OF THE INVENTION

The invention relates generally to an apparatus and method for the guided penetration of a chronic total occlusion (CTO) in a blood vessel and, more particularly, to the use of an ultrasound-based detection system to direct catheter and drill placement during penetration of an occlusion.

### BACKGROUND

One of the leading causes of human mortality is cardiovascular disease. This commonly begins with stenotic lesions of the coronary arteries, which occur, for example, as a result of the gradual buildup of atheromata, or plaques, along the vessel walls. This buildup leads to a gradual reduction in the diameter of the lumen over time and the subsequent restriction of blood flow. A chronic total occlusion results when a blood vessel becomes completely occluded by plaques for an extended period of time. Such occlusions occur not only in coronary arteries but in other blood vessels as well. A CTO may contain soft plaques, but not infrequently a CTO develops hard plaques which comprise dense, fibrous tissue and calcification at the proximal and distal ends. Until recently, the most common method of treating CTO was bypass surgery, which is a procedure that, unfortunately, involves considerable risk and trauma to the patient.

Advances in modern medicine have led to the development of recanalization procedures for treating such obstructive vascular disorders, for example, balloon angioplasty, atherectomy and stent implantation. These procedures typically require the initial insertion and placement of a guide wire across the occluded region. The guide wire is percutaneously inserted into the blood vessel carrying an interventional catheter, it is directed to and through the stenoses that form the occlusion. The interventional catheter carries, e.g., a balloon and/or stent used to open the occlusion. The primary purpose of the guide wire is to provide an accessible rail over which the physician can route the interventional catheter and subsequently recanalize the occluded lumen using one or more of the aforementioned treatment procedures.

While CTO containing soft plaques are often amenable to penetration with a guide wire, CTO containing hard plaques are often difficult to penetrate successfully with a guide wire, especially where the lesion is heavily calcified. Such situations introduce additional and highly undesirable complexity to the procedure, requiring removal of the guide wire and reinsertion of a stiffer wire. Further, when the tip of the guide wire encounters and fails to penetrate the CTO, it can veer towards the wall of the vessel, thereby possibly damaging, or worse, perforating the vessel wall and other times forming a false lumen.

Some methods have been developed to recanalize this more difficult type of occlusion. In U.S. Pat. No. 5,935,108, a needle cannula is mounted within the lumen of a guiding sheath. The needle cannula is used to recanalize the occluded vessel with the help of a guide wire that has an ultrasonic transducer mounted on its distal end to permit the operator to determine whether the occlusion is present or has been successfully crossed, using Doppler shifts in ultrasonic waves. Similarly, U.S. Pat. No. 5,938,671 discloses a device that uses a sharpened tip along with two-dimensional ultrasound-based imaging. U.S. Pat. Nos. 6,611,458, 6,221,049 and 6,217,527 disclose a method of bypassing a CTO in which a guide wire is redirected through the subintimal space formed between the intimal and adventitial layers of the blood vessel wall. These methods have the disadvantage of only providing a two-dimensional image and of not being able to precisely determine the position of the catheter's distal end relative to the occlusion.

One approach that promotes CTO penetration involves ultrasonic angioplasty. U.S. Pat. Nos. 6,482,218 and 5,304,115 describe a wire-shaped ultrasonic catheter that is used to penetrate through hard, calcified deposits of atheroma by pneumatic drilling and through non-calcified material by cavitation. While these patents describe a method of penetrating the CTO, they do not disclose a way to accurately direct the placement of the catheter during the procedure.

The successful penetration of an occlusion in a vessel, particularly a CTO, is heavily dependent upon the ability to monitor, in real time, the position of the distal tip of the guide wire or drilling tip as it is advanced through the lesion. Therefore, there is a need in the art for an apparatus that permits accurate positioning of the guide wire and also facilitates the penetration of a CTO—in coronary blood vessels as well as other types of vessels—and for methods of using such an apparatus. Such an apparatus and method would reduce the risk of penetrating the blood vessel or creating a false lumen.

Accordingly, it is an object of the present invention to provide an apparatus comprising a guide wire having a vibrating end or a tip, in particular a therapeutic tip, that vibrates at a frequency sufficient to penetrate and traverse a vessel occlusion, while simultaneously providing a detectable vibration frequency that permits locating the therapeutic tip, e.g., relative to the body lumen, thereby enabling an operator to direct the guide wire towards a lesion or through an occlusion in a vessel and away from the vessel walls. Another object of the invention is to provide a method of treating a CTO by facilitating penetration of the CTO while simultaneously visualizing the procedure.

### SUMMARY OF THE INVENTION

The invention is laid down in the attached claims, wherein advantageous embodiments thereof are subject-matter of the dependent claims, respectively. The present invention generally relates to an apparatus that facilitates accurate placement of guide wire and drilling tip within the blood vessel during recanalization of a CTO or lesion and a method of using such an apparatus. The apparatus comprises a detection system that permits differential processing of ultrasonic frequency signals from the guide wire, occlusion and vessel walls using one or more signal receivers that can be oriented at different angles with respect to the area being treated. Preferably, the detection system is an imaging system, more preferably the imaging system is capable of generating real-time three-dimensional images of the CTO or lesion, the blood vessel and the distal tip of the guide wire. Preferably, the apparatus also facilitates penetration of a CTO or other obstructions in a vessel. The method of using the apparatus of the invention facilitates treatment of the occlusion and avoids or reduces the complications and risks associated with treating an occlusion, such as perforation of the walls of the vessel and creation of a false lumen.

The guide wire of the apparatus has a tip, in particular a therapeutic tip, at its distal end, capable of vibrating at user-definable frequencies, including ultrasonic frequencies, that are detectable by the detection or imaging system. The tip or therapeutic tip at the distal end of the guide wire is further capable of vibrating at a frequency sufficient to enable the therapeutic tip of the guide wire to drill through a vessel occlusion. These features may be accomplished by several combinations of particular elements, but are summarized by the non-limiting embodiments described below.

One embodiment relates to a system comprising a drilling component for use in penetrating an occlusion in a body lumen coupled to an imaging component that permits visualization of the drilling procedure resulting in lower risk and fewer complications. The power source and controller work together to energize the transducer causing the therapeutic tip to vibrate or oscillate at a desired frequency. The controller permits operation of the transducer over a range of frequencies and amplitudes, including at frequencies and amplitudes that are useful in drilling and frequencies and amplitudes that are detectable by the receivers. The controller, power source, transducer, therapeutic tip and catheter work together to generate detectable signals from the body lumen and, with the one or more receivers and an imaging system, generate real-time images of the distal end of the guide wire and catheter relative to the walls of the body lumen and the occlusion. This permits the operator to visualize the therapeutic tip and to guide the therapeutic tip and guide wire through the occlusion and away from the vessel walls.

A preselected range of vibrational frequencies generated by the energy-generating system facilitates recanalization of an occluded vessel by energizing an oscillating ceramic motor to a vibrational frequency and amplitude, in particular a vibrational frequency and amplitude sufficient for the therapeutic tip to operate as a drilling device, e.g., to penetrate the occlusion. The oscillating ceramic motor is also made to vibrate at a frequency detectable by the imaging system. In a preferred embodiment, the energy generating system energizes the oscillating ceramic motor to vibrate at an ultrasonic frequency.

Another embodiment relates to a method of imaging a guide wire during penetration of a vessel occlusion to direct the guide wire more accurately during drilling. The method comprises introducing a guide wire having a tip, in particular a therapeutic tip, a catheter and a functionally connected transducer into a blood vessel having an occlusion and advancing said catheter and guide wire through said blood vessel until the therapeutic tip on the distal end of the guide wire contacts the proximal end of the occlusion; activating a power source to energize the transducer so as to vibrate the therapeutic tip of the guide wire at a frequency sufficient to penetrate the occlusion and to generate detectable signals from the therapeutic tip, obstruction and vessel walls; collecting the detectable signals using one or more receivers, said receivers connected to a controller which is capable of processing the information from the receivers and generating a real-time image on an image screen; using the images on the image screen to guide the therapeutic tip through the occlusion and away from the vessel walls. In some applications, one or more passes may be utilized to clear the occlusion. In a preferred aspect of the embodiment, the frequency of the detectable signals is in the ultrasonic range and the receivers are capable of detecting the ultrasonic signals.

### BRIEF DESCRIPTION OF THE DRAWINGS

FIG. 1 depicts an image-based guiding system of the invention.

FIG. 2 depicts an axial cross-section of the distal end of the catheter and guide wire from an embodiment of the system depicted in FIG. 1.

FIG. 3 depicts an axial cross-section of the distal end of the catheter and guide wire from another embodiment of the system depicted in FIG. 1.

FIG. 4a depicts a transverse section through the catheter of an embodiment of the invention at a point such as 4A in FIG. 2, showing a plurality of lumens and components therein. FIG. 4b depicts a transverse section through the catheter of an embodiment of the invention at a point such as 4B in FIG. 2, showing a cylindrical transducer attached to the catheter and the guide wire positioned in the bore of the transducer.

FIG. 5a depicts the embodiment of FIG. 2 within a blood vessel having an occlusion; FIGS. 5b and 5c show portions of an image-based guiding system of the invention in use, at a point of partial penetration of a blood vessel occlusion.

### DETAILED DESCRIPTION

The present invention generally provides a system for use in guiding an apparatus through a body lumen—such as intravascularly—with accuracy, so as to avoid or reduce the risk of perforating the lumen wall or creating a false lumen. The system comprises a flexible, elongated catheter having a proximal and distal end with at least one lumen extending longitudinally therethrough; a guide wire, also having a proximal and distal end and further having a tip, e.g., a therapeutic tip, at its distal end; a transducer capable of being energized to vibrate at a detectable frequency, which in turn causes the therapeutic tip to vibrate; a power source; a controller for controlling the power source; and a detection system. The detection system comprises one or more receivers and utilizes the controller, which further comprises a processor, for converting signals detected by the receivers into differentiable information. Preferably, the detection system is an ultrasound-based imaging system, comprising an image screen.

The presence of an occlusion in a blood vessel has the deleterious effect of restricting blood flow and affecting the health of the patient. A typical treatment procedure involves percutaneous introduction of a guide wire into a blood vessel and directing the guide wire through the vessel to the occlusion site. According to the present invention, a transducer vibrates the therapeutic tip of the guide wire, enabling the therapeutic tip to penetrate the hard mass of the occlusion. Penetration and traversal of the occlusion may be effected by vibrational drilling and/or cavitation. Also according to the present invention, when the transducer is activated to vibrate at an ultrasonic frequency, one or more receivers capable of ultrasound-based detection of these ultrasonic vibrations can be used to locate and guide the placement of the therapeutic tip. Thus, for example, by energizing an OCM, e.g., on or at the distal end of the catheter and/or guide wire at appropriate frequencies, it is possible not only to drill through the occlusion allowing the guide wire to penetrate the occlusion but also to guide the therapeutic tip in a manner that avoids perforation of the vessel walls or creation of a false lumen using images generated from vibration frequencies detected by one or more external receivers.

The guide wire is slideably mounted on the catheter and its therapeutic tip comprises, or is functionally coupled to, the transducer. The guide wire's total length typically may be greater than that of the catheter such that its proximal end and distal end extend beyond the proximal end and distal end, respectively, of the catheter. In one embodiment, the transducer is preferably a piezoelectric motor, more preferably a miniature oscillating ceramic motor, which is energized from the power source. The energized transducer causes the therapeutic tip of the guide wire to vibrate at a drilling frequency, so that the vibrating therapeutic tip is capable of drilling through the occlusion. The energized transducer also generates a signal of detectable frequency in the therapeutic tip. When the therapeutic tip contacts the surface of an occlusion in the body lumen, it causes the immediate contact area of the occlusion and adjacent tissue, such as walls of the body lumen, to vibrate at a detectable frequency. The power source is functionally connected to the controller, which controls the amount of energy the power source sends to the transducer, and thus the frequency of vibration of the therapeutic tip. In the embodiment in which the detection system is an imaging system, the receivers detect signals produced by the vibrating therapeutic tip, the vibrating occlusion and surrounding tissues and transmit those signals to a processor which generates an image of the therapeutic tip, occlusion and surrounding tissues. The processor may generate real-time images on the image screen. The images may be 3-dimensional. The controller and processor may be connected components or may be the same component, in which case the component may be, in part, a computer—for example, a laptop or desk computer having software to control the power source and to process electrical signals from the receivers into images.

Miniature Oscillating Ceramic Motors (OCM) are known in the art and are disclosed in U.S. Pat. No. 5,453,653 to Zumeris, the specification as it relates to OCMs is incorporated herein by reference. These motors can be made very small and in any shape, and their small size and low energy level requirements make them especially suitable for use inside living organisms. They operate by contacting a surface in an amount sufficient to generate sufficient friction to permit the motor to “crawl” along the contacted surface and change its position relative to the contacted surface when the motor is energized or by vibrating an attached component or the contacted surface. In the present case, for example, an OCM can be made to vibrate the therapeutic tip of the guide wire to facilitate the wire passing through a vessel occlusion. An OCM also can be made to vibrate the therapeutic tip at a detectable frequency. Alternatively, an OCM may vibrate the therapeutic tip at detection frequencies and another vibration transducer may vibrate the therapeutic tip at drilling or cavitation frequencies. OCMs can be adequately insulated to act in aqueous environments. A ceramic motor used in accordance with the present invention may cause the guide wire to “drill” through the calcified or fibrous parts of the occlusion, which are impenetrable by ordinary guide wires. The frequencies utilized in the various embodiments described herein may be varied as specific embodiments may require. A wide range of frequencies, e.g., radio frequency (rf) or ultrasound (us), may be utilized depending upon the type and the location of the tissue being treated and the type and amount of tissue through which the vibrations must pass.

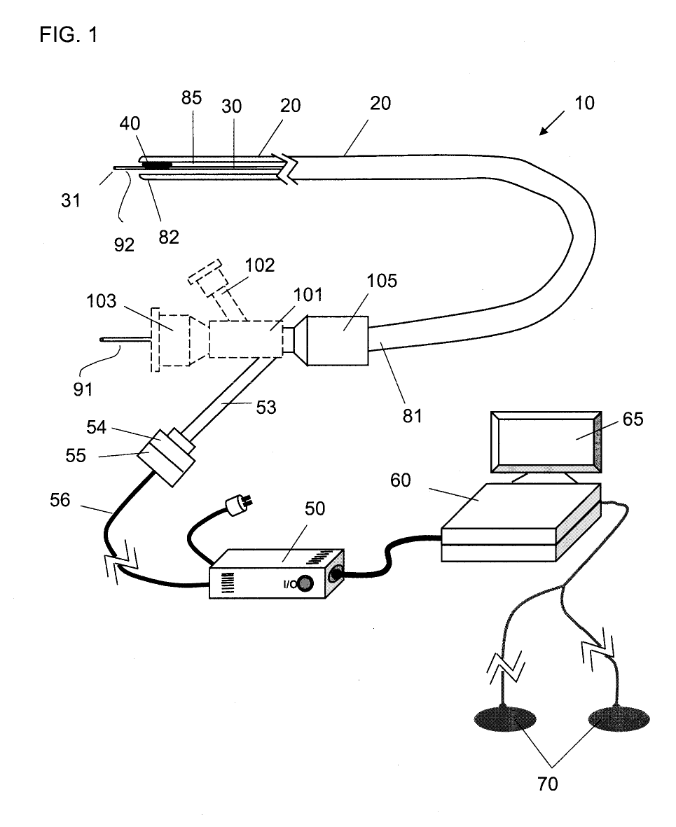

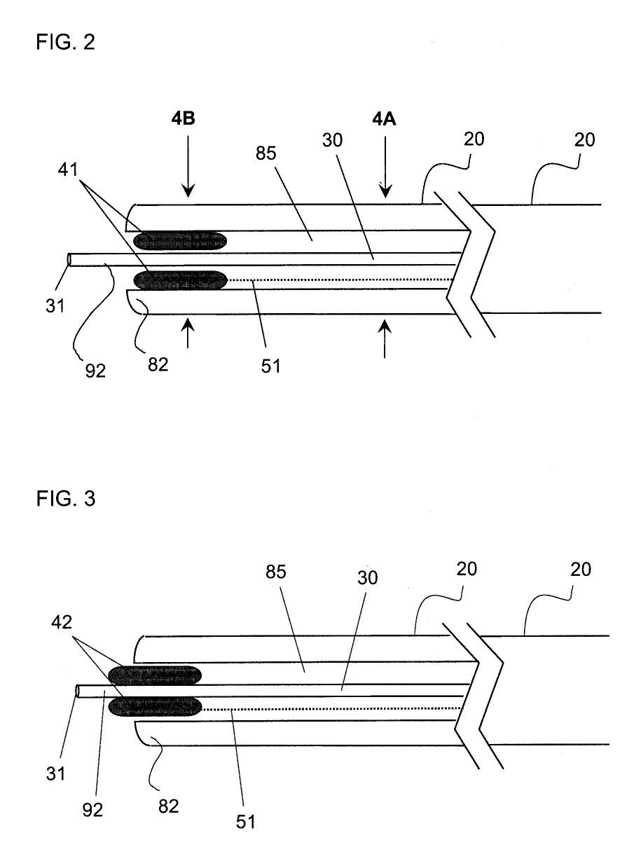

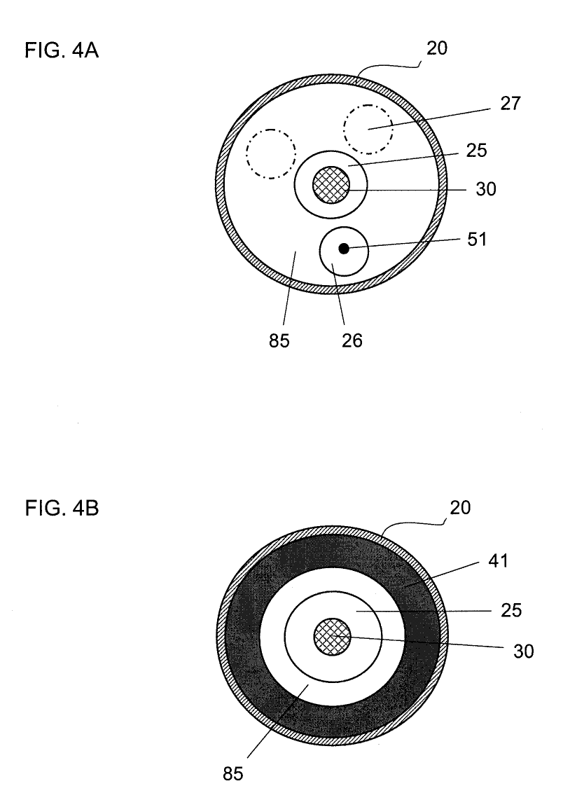

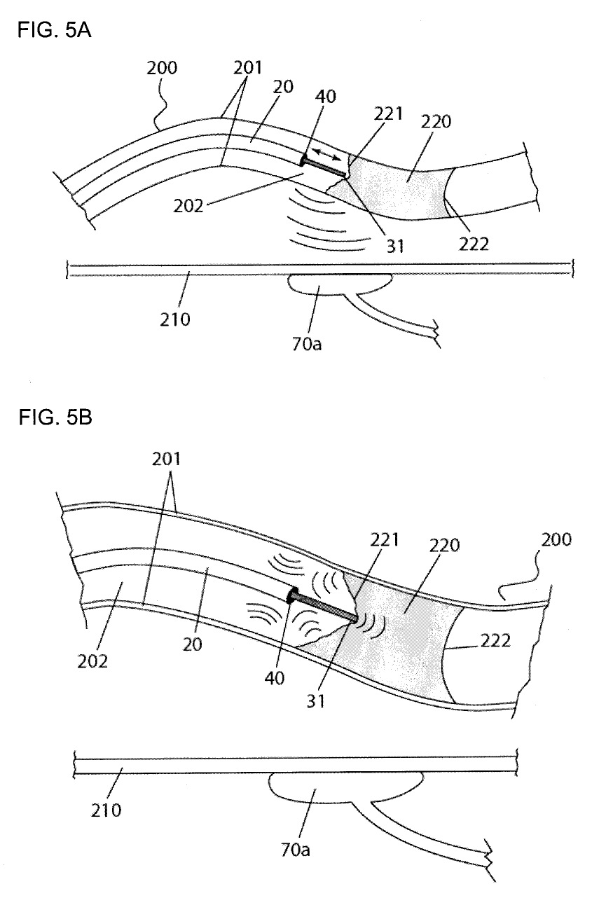

As shown in particular in FIG. 1, a preferred embodiment of the system 10 comprises a catheter 20 having a proximal end 81 and a distal end 82 and a lumenal space 85 therebetween, the lumenal space 85 containing at least one lumen (see FIG. 4a); a guide wire 30 within a lumen 25 (not shown in FIG. 1) of the lumenal space 85 of the catheter 20, the guide wire 30 having a proximal end 91 and a distal end 92 and a therapeutic tip 31 at its distal end 92; a transducer 40 (embodiments of which are shown in FIGS. 2 and 3 as 41 and 42, respectively); a power source 50; and an imaging system comprising a controller 60; one or more receivers 70; and an image screen 65. The controller 60 processes signals collected by the receivers 70 to create images. Preferably, the controller 60 comprises a computer and software to process, and optionally save, the images. Preferably, the guide wire 30 is longer than the catheter 20, such that at least its distal end 92 extends beyond the distal end 82 of the catheter 20. Preferably, the tip of the guide wire 30 is made sufficiently hard and the guide wire itself is sufficiently stiff to penetrate a vessel occlusion (e.g., all commercial guide wires classified as stiff or extra-stiff), yet sufficiently flexible to navigate tortuous blood vessels, such as coronary vessels. The catheter 20 may be any endovascular catheter known in the art, including interventional catheters for delivering endovascular devices, such as stents, or for other therapeutic uses, such as angioplasty.

In addition to processing signals from the receiver 70, the controller 60 interfaces with the power source 50 and is capable of controlling the amount of energy emitted from said power source 50. Preferably the power source 50 is a combined sonic and ultrasonic power source such that optimization can be made separately for occlusion penetration frequencies (sonic) and imaging frequencies (ultrasonic) to produce a combined vibration. The controller 60 also interfaces with one or more receivers 70 and the image screen 65. In one embodiment, the controller 60 is a laptop or desktop computer containing software that controls the power source 50 and processes signals that may be captured by the receivers 70 into a real-time 3-dimensional image on the image screen 65. The power source 50 generates energy that is transmitted to the transducer 40 and energizes the transducer 40. Preferably the transducer 40 is a piezo-electric motor; more preferably the piezo-electric motor is an OCM. But, for purposes of generating the image, the source of vibration both for drilling and for imaging may be any other vibrating source.

According to the embodiment depicted in FIG. 2, the transducer 41 is located at the distal end 82 of the catheter 20 and is capable of transferring vibration energy to the distal end 92 of the guide wire 30 near the therapeutic tip 31. In another embodiment, depicted in FIG. 3, the transducer 42 is located on the therapeutic tip 31 of the guide wire 30.

When the transducer 40 is a piezoelectric motor or OCM, it is made of a suitable material such as ceramic, quartz or other suitable materials known in the art. The piezoelectric motor is capable of receiving electrical energy and converting it to mechanical energy in the form of longitudinal motion or vibrational wave pulses. The mechanical energy can be used to aid penetration of the occlusion through cavitation and/or pneumatic drilling. The frequency and amplitude of vibration generated by the transducer can be varied by the controller 60 such that parameters suitable for ultrasonic imaging and more effective drilling can be realized. Alternatively, the apparatus may comprise more than one transducer, of the same type or of different types. For example, the apparatus may comprise two transducers, each energized by the power source 50. One transducer may be energized to vibrate the therapeutic tip at a penetration frequency, the other transducer may be energized to vibrate the therapeutic tip at an imaging frequency.

The piezoelectric motor may be energized by either an AC or DC source, but preferably it is energized by an AC source. When the piezoelectric motor is an OCM, the frequency of the AC energy input to the OCM will cause the OCM to vibrate in the range of 20-100 kHz, the oscillation depending on the resonant frequency of the material used for the piezoelectric ceramic. When the OCM is energized in a DC pulsed mode, two electrodes are excited by positive voltage and two electrodes are excited by negative voltage. The left side of the energized OCM becomes longer than the right side and the OCM moves to the right, thereby moving the therapeutic tip of the guide wire to the right. When the voltage is stopped, the OCM will move back to its original position. The oscillation (vibrating or pulsating motion) will occur at a frequency dependent on pulse time, preferably 10-50 msec., or a pulsating frequency of 20-100 kHz.

The transducer 40 (and 41, 42) is capable of receiving energy from the power source 50, preferably from electrical conducting wires 51 (shown in FIGS. 2, 3 and 4a) that connect the transducer 40 directly or indirectly to the power source 50. As depicted in FIG. 4, said electrical conducting wires 51 may lie within a lumen 26 of the lumenal space 85 of the catheter 20, other than the lumen 25 in which the guide wire 30 lies. The electrical conducting wires 51, 52 extend the entire length of the catheter 20 past its proximal end 81. In FIG. 1, said electrical conducting wires 51 (not shown) are connected to the power source 50. For example, electrical conducting wires 51 (see FIGS. 2, 3 and 4a) exit the catheter 20 at a fitting 105, which is connected to a hub 101 where the electrical conducting wires 51 connect to a cord 53, which runs from the hub 101 to a first connector 54. The first connector 54 is pivotally coupled to a second connector 55; an electrical cable 56 connects said second connector 55 to the power source 50. Said cord 53 and said cable 56 contain conductors (not shown) to transmit energy from the power source 50 to the electrical conducting wires 51.

In an alternative embodiment (not shown), the transducer 40 further comprises a sensor that is capable of receiving energy remotely from a power source and energizing the transducer 40. In this embodiment, electrical conducting wires 51, cord 53, or electrical cable 56 are not required. Specifically, the sensor is adapted to communicate with a power source for selectively generating and transmitting ultrasonic vibrations, such that it receives the transmitted energy and transfers the energy to the transducer, energizing the transducer.

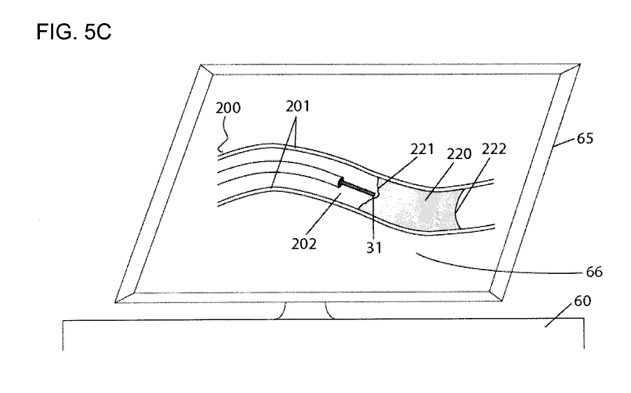

As depicted in FIG. 5a, the catheter 20 and guide wire 30 are introduced into a body lumen, such as a blood vessel 200. The blood vessel 200 has a vessel wall 201 and a lumen 202, which lumen 202 is blocked by an occlusion 220. The occlusion 220 has a proximal cap 221 and a distal cap 222. The occlusion 220 is expected to comprise fibrous and/or calcified material (not shown), with a higher proportion of calcified material at its proximal and distal caps 221, 222. The therapeutic tip 31 of the guide wire 30 is placed near the proximal cap 221 of the occlusion 220. The power source 50 transmits sufficient energy to the transducer 40 to cause the transducer 40 to vibrate the therapeutic tip 31 at a frequency sufficient to penetrate the harder material of the proximal cap 221 of the occlusion 220. Specifically, the energized transducer causes the therapeutic tip 31 of the guide wire 30 to vibrate, such that—when in a blood vessel 200 having an occlusion 220 and placed in contact with the proximal cap 221 of the blood vessel occlusion 220—the vibrating therapeutic tip 31 is capable of functioning as a drill to penetrate said occlusion 220.

During the procedure of penetrating the occlusion 220, the energized vibrating therapeutic tip 31 is placed at the proximal cap 221 of the occlusion 220 and is made to penetrate the occlusion 220. The high frequency vibration of the vibrating therapeutic tip 31 permits the therapeutic tip 31 to act like a drill, enabling the distal end 92 of the guide wire 30 to penetrate the occlusion 220, as depicted in FIG. 5b.

In the embodiment depicted in FIG. 2, the catheter-mounted transducer 41 is attached to the distal end 82 of the catheter 20, but functionally communicates with the therapeutic tip 31 of the guide wire 30. In this embodiment, the catheter-mounted transducer 41 is cylindrical with a bore through the middle and fits within the lumenal space 85 at the distal end 82 of the catheter 20. The cylinder bore forms a guide wire lumen, through the center of which the guide wire 30 passes. As shown in FIGS. 5a-5c, after the guide wire 30 and catheter 20 are advanced to the proximal cap 221 of the occlusion 220, the catheter 20 is secured and the catheter-mounted transducer 41 is energized, causing the catheter-mounted transducer 41 to vibrate the therapeutic tip 31 of the guide wire 30. In operation, the catheter-mounted transducer 41 is capable of communicating with the therapeutic tip 31 moving the tip in an oscillatory manner that causes the therapeutic tip 31 to vibrate, or move, and function as a drilling device against a lumenal obstruction such as a blood vessel occlusion 220 or CTO. In an alternative arrangement (not shown), the transducer may be one or more slab-shaped transducer(s) disposed on the inner wall of the catheter 20. The transducer is adapted to frictionally engage the guide wire 30 and move the guide wire 30 relative to the catheter 20.

In the embodiment depicted in FIG. 3, the guide wire-mounted transducer 42 is attached to the therapeutic tip 31 of the guide wire 30. In this embodiment, the guide wire-mounted transducer 42 is located at the distal end 82 of the catheter 20 and may be attached to the distal end 82 of the catheter 20, so as to anchor the guide wire-mounted transducer 42 and therapeutic tip 31 during operation. FIGS. 2 and 3 illustrate two non-limiting embodiments of a transducer and its arrangement relative to the catheter and guide wire. Other possible arrangements are within the skill in the art. For example, the transducer 40 may be located more remotely from the therapeutic tip 31, provided that the catheter 20 is designed to permit vibration for drilling and vibration for detection to be transmitted to the therapeutic tip 31.

In addition to energizing the transducer 40 to vibrate the therapeutic tip 31 for drilling through the occlusion 220, the power source 50 provides vibrational energy for detection purposes. Specifically, the power source 50 also transmits energy to the transducer 40 sufficient to generate a vibrational frequency, preferably an ultrasonic or sonic frequency, more preferably an ultrasonic frequency, which frequency is transmitted to the therapeutic tip 31 and adjacent bodily tissues to create detectable signals from the vibrating therapeutic tip 31 and surrounding tissues. These signals may be collected by one or more receivers 70, as shown for one receiver 70a in FIGS. 5a and 5b.

FIGS. 5a-5c depict an embodiment of the invention in use. The operator places the energized vibrating therapeutic tip 31 in contact with the proximal cap 221 of the occlusion 220, and the operator maintains contact between the therapeutic tip 31 and some portion of the occlusion 220 throughout the drilling process, as shown in FIGS. 5a and 5b. When energized to vibrate at a detectable frequency, this contact permits transfer of detectable vibrations to the occlusion 220 and vessel walls 201, which deflect detectable signals to one or more receivers 70, as depicted with one receiver 70a in FIGS. 5a and 5b. Energizing the transducer 40 also causes the transducer 40 to vibrate the therapeutic tip 31 at a frequency capable of generating detectable signals, which are collected by one or more receivers 70. The one or more receivers 70 are functionally connected to the controller 60 and image screen 65 (see FIG. 1), such that the signals—in particular from the therapeutic tip, occlusion, vessel walls and surrounding tissues—can be detected by the one or more receivers 70 and may be differentially processed into 3-dimensional images 66 on the image screen 65.

Specifically, the one or more receivers 70, when placed against the body wall of the patient 210 as exemplified for one receiver 70a in FIGS. 5a and 5b, are capable of receiving signals from vibrations of a detectable frequency, preferably a sonic or ultrasonic frequency. The vibrating therapeutic tip 31 produces detectable signals, and transmits vibrations to the occlusion 220 and blood vessel walls 201, thereby producing detectable signals from those structures. These detectable signals are received by one or more receivers 70 and transmitted to the controller 60, where they are processed. In this way, the position of the vibrating therapeutic tip 31 relative to the occlusion 220 and blood vessel walls 201 may be visualized on the image screen 65 as 3-dimensional images 66, as depicted in FIG. 5c.

The system of the invention, as depicted in FIG. 5c, provides real-time feedback in the form of a 3-dimensional image 66 as to the position of the therapeutic tip 31 while drilling into and through the occlusion 220. This allows the operator to adjust the position of the drilling therapeutic tip 31 and direct the guide wire 30 through the occlusion 220 and away from blood vessel walls 201 to avoid perforating said blood vessel walls 201.

In an alternative embodiment, the signals—in particular from the therapeutic tip, occlusion, vessel walls and surrounding tissues—are detected by the one or more receivers 70 and may be differentially processed into numerical information that can be transformed into a non-image form perceptible by the operator. Specifically, the detectable signals, which are transmitted to a controller 60 comprising a processor, are processed as information that may be transformed by the controller in any manner known in the art into parameters useful to the operator—such as linear scans, numerical output, audible signals or other parameters. The system of the invention thereby can provide real-time feedback in the form of non-image-based information as to the position of the therapeutic tip 31 while drilling into and through the occlusion 220. In this way, the position of the vibrating therapeutic tip 31 of the guide wire 30 relative to the occlusion 220 and blood vessel walls 201 may be used by the operator to adjust the position of the drilling therapeutic tip 31 and direct the guide wire 30 through the occlusion 220 and away from blood vessel walls 201 to avoid perforating said blood vessel walls 201.

Optionally, the hub 101, shown in FIG. 1, can carry any number of suitable or necessary members useful for intravascular procedures. For example the hub 101 may have a flush port 102 through which a suitable flushing or cooling liquid such as saline solution can be introduced, or an assembly comprising a hemostasis valve 103 through which the guide wire 30 and conducting wires 51, 52 may extend.

Also provided are methods of using the apparatus of the invention for penetrating or recanalizing a vessel occlusion. One such method comprises a) providing a device comprising: i) a guide wire having a proximal end, a distal end and a therapeutic tip at said distal end; ii) a catheter having a proximal end, a distal end, and a longitudinal bore therethrough; iii) a piezoelectric micromotor, said micromotor capable of generating one or more vibrational frequencies when energized by a power source and capable of causing said therapeutic tip to vibrate at said one or more vibrational frequencies; iv) an imaging system comprising one or more receivers for receiving vibrational frequency signals and an imaging screen; b) introducing said guide wire into a blood vessel having vessel walls and an obstruction, and advancing said guide wire until said therapeutic tip of said guide wire contacts said obstruction, wherein said catheter is slideably mounted on said guide wire, said guide wire passing through said longitudinal bore of said catheter; c) advancing said catheter over said guide wire until said distal end of said catheter meets said obstruction, said micromotor now being operatively coupled to said therapeutic tip; d) energizing said piezoelectric micromotor so that said therapeutic tip advances distally through said obstruction in an oscillating or vibrating manner; e) generating detectable vibrational frequency signals from said vibrating therapeutic tip, obstruction and vessel walls via said piezoelectric micromotor; f) detecting said vibrational frequency signals with said one or more receivers of said imaging system, and using said imaging system to generate real-time images of said therapeutic tip relative to said obstruction and said vessel walls; and g) using said generated images to direct said guide wire through said obstruction and away from said vessel walls. The vibration transducer may be a piezoelectric micromotor.

In particular a method for guiding an endovascular device through a blood vessel occlusion is provided. The method comprises a) providing a device, said device comprising: i) a guide wire having a proximal end, a distal end and a therapeutic tip at said distal end; ii) a catheter having a proximal end, a distal end, and a longitudinal bore therethrough; iii) a piezoelectric micromotor, said micromotor capable of generating one or more vibrational frequencies when energized by a power source and capable of causing said therapeutic tip to vibrate at said one or more vibrational frequencies; iv) an imaging system comprising one or more receivers for receiving vibrational frequency signals and an imaging screen; b) introducing said guide wire into a blood vessel having vessel walls and an obstruction, and advancing said guide wire until said therapeutic tip of said guide wire contacts said obstruction, wherein said catheter is slideably mounted on said guide wire, said guide wire passing through said longitudinal bore of said catheter; c) advancing said catheter over said guide wire until said distal end of said catheter is in close proximity of said obstruction, said micromotor now being operatively coupled to said therapeutic tip; d) energizing said piezoelectric micromotor so that said therapeutic tip penetrates said obstruction in an oscillating or vibrating manner; e) generating detectable vibrational frequency signals from said vibrating therapeutic tip, obstruction and vessel walls via said piezoelectric micromotor; f) detecting said vibrational frequency signals with said one or more receivers of said imaging system, and using said imaging system to generate real-time images of said therapeutic tip relative to said obstruction and said vessel walls; and g) using said generated images to direct said guide wire through said obstruction and away from said vessel walls.

In some embodiments, one or more passes of the guide wire through the obstruction may be necessary to clear the blood vessel lumen of obstructive material. To assist penetration of a vessel occlusion by repositioning the guide wire and/or advancing the guide wire and catheter through the occlusion as it is recanalized, the apparatus may further comprise a piezoelectric micromotor designed and placed to permit movement of the catheter relative to the guide wire, as described in U.S. Pat. No. 6,238,401, which is incorporated herein by reference. Such an apparatus may comprise a catheter having a proximal end and a distal end and a longitudinal bore therethrough; a guide wire having a proximal end and a distal end and a therapeutic tip at said distal end; a piezoelectric micromotor; a power source for energizing said piezoelectric micromotor causing said piezoelectric micromotor to vibrate at a first frequency, said piezoelectric micromotor being functionally connected to said therapeutic tip so as to vibrate said therapeutic tip at said first frequency, and for energizing said piezoelectric micromotor at a second frequency, said second frequency being sufficient to create detectable signals; a controller connected to said power source for controlling energy supplied to said piezoelectric micromotor from said power source and thereby control said first frequency; a detection system comprising one or more receivers for collecting said detectable signals and a controller comprising a processor for transforming said signals into differentiable information; wherein said differentiable information may include relative positions of said therapeutic tip, said obstruction and said body lumen, wherein said body lumen has lumen walls, and wherein said detection system permits an operator to use said differentiable information to position said therapeutic tip relative to said body lumen walls and said obstruction.

Methods of using such an apparatus are further provided. One such method comprises advancing said guide wire and said catheter distally by—repeatedly, until said guide wire and said catheter pass substantially through said obstruction—(i) securing said catheter; (ii) releasing said guide wire and energizing said pulling motor so that said guide wire advances distally; (iii) securing said guide wire; (iv) releasing said catheter and energizing said pulling motor so that said pulling motor advances along the guide wire, carrying with it said catheter. Another such method for recanalizing an occlusion, wherein the apparatus comprises a piezoelectric crawling motor capable of pulling said catheter along said guide wire, comprises alternately (i) energizing said crawling motor so that said guide wire advances distally; (ii) energizing said crawling motor so that said guide wire advances proximally; (iii) repeating steps (i) and (ii) a plurality of times until said guide wire has substantially recanalized said obstruction.

In related embodiment, the same piezoelectric micromotor that enables penetration of the occlusion may also move the guide wire and catheter through the occlusion. The method of using such an apparatus comprises a) percutaneously inserting into a body lumen having a target area containing an obstruction an apparatus comprising a cylindrically shaped motor attached to said device, said motor having a longitudinal bore, said motor provided with a motor friction area disposed within said longitudinal bore, a guide wire disposed within said longitudinal bore, said guide wire and said longitudinal bore of said motor sized and adapted to impart friction between said friction area of said motor and said guide wire in an amount sufficient to permit said motor to change position relative to said guide wire by crawling against said guide wire when said motor is energized; b) advancing said guide wire to said target area; c) securing said guide wire; d) energizing said motor so that said motor vibrates and advances along said guide wire to said target area to drill through said obstruction to clear said obstruction from said target area of said lumen; e) vibrating said therapeutic tip, obstruction and walls of said lumen at an ultrasonic frequency, said vibration generating detectable signals; f) collecting said detectable signals and imaging said guide wire, obstruction and walls of said lumen in real time; and g) directing said guide wire through said obstruction and away from said walls of said lumen. In any of the methods of the invention, the catheter may be mounted onto the guide wire after advancing the guide wire to the vessel occlusion, or the catheter may be mounted onto the guide wire prior to advancing the guide wire to the vessel occlusion. In the latter case, the step of advancing the catheter to close proximity of the occlusion may comprise adjusting the position of the catheter on the guide wire relative to the occlusion, such that an operable amount of guide wire extends distal of the distal end of the guide wire.

The image-guided system of the invention may be used to guide the drilling therapeutic tip of the guide wire through the occlusion to widen the passageway sufficiently, for example, to deploy a therapeutic device such as a balloon or stent. Where deployment of a therapeutic device is desired, the catheter 20 may be an interventional catheter carrying a therapeutic device such as an angioplasty balloon or a balloon expandable or self-expanding stent, or the catheter 20 may be exchanged with a second interventional catheter carrying the desired therapeutic device. If such a therapeutic device is to be deployed, the catheter 20 or interventional catheter is advanced through the cleared blood vessel once the guide wire 30 has sufficiently cleared the occlusion 220.

It will be appreciated by persons having ordinary skill in the art that many variations, additions, modifications, and other applications may be made to what has been particularly shown and described herein by way of embodiments, without departing from the spirit or scope of the invention. Therefore it is intended that scope of the invention, as defined by the claims below, includes all foreseeable variations, additions, modifications or applications.

1-25. (canceled)

26. An intravascular catheter system for penetration and imaging of an occlusion, comprising:
a therapeutic tip;
a first vibration transducer connected to said therapeutic tip, said first vibration transducer capable of vibrating said tip at a first frequency sufficient to penetrate an occlusion, said occlusion located in a vessel having walls;
a second vibration transducer, capable of generating a detectable signal at a second frequency, said second frequency different than said first frequency and transmitted simultaneously therewith, wherein said detectable signal may be processed into an image of at least one of said tip, occlusion and vessel walls, to enable said tip to be positioned relative to said occlusion and said vessel walls; and
a power source, said power source being capable of energizing said first and second vibration transducers.

27. The system of claim 26 wherein said power source is connected to a controller.

28. The system of claim 27, further comprising detection and imaging components, including
a receiver for collecting said detectable signal; and
an image screen;
a processor for transforming said signals into images.

29. The system of claim 27, further comprising detection components, including a receiver for collecting said detectable signals;
and further comprising a processor for transforming said signals into differentiable information of non-image form, said differentiable information including relative positions of said therapeutic tip, said walls and said obstruction.

30. The intravascular catheter system according to claim 26, wherein the therapeutic tip is a drilling tip.

31. The intravascular catheter system according to claim 30, wherein the drilling tip comprises a rigid material.

32. The intravascular catheter system according to claim 31, wherein the drilling tip comprises a metal.

33. The intravascular catheter system according to claim 26, wherein said detectable signal has a frequency in the acoustic range selected from the group consisting of ultra sound range, sonic range and infrasonic range.

34. The intravascular catheter system according to claim 26, wherein the detectable signal is transmitted at a range of frequencies.

35. The intravascular catheter system according to claim 34, wherein said range of frequencies is selected from the group consisting of ultra sound range, the sonic range and the infrasonic range.

36. The intravascular catheter system of claim 26, wherein said first frequency is in the range selected from the group consisting of ultra sound range, sonic range and infrasonic range.

37. The intravascular catheter system of claim 26, wherein said detectable signal is generated from outside the body.

38. The intravascular catheter system of claim 26, wherein said detectable signal is transmitted from outside the body.

39. The intravascular catheter system of claim 28, wherein said generated image is a Doppler image.

40. The intravascular catheter system of claim 28, wherein said generated image is a 3D image.

41. The intravascular catheter system of claim 28, wherein said generated image may include images of the group consisting of the occlusion, therapeutic tip and the vessel proximal or distal to said occlusion.

42. The intravascular catheter system of claim 26, wherein the first vibration transducer is a piezoelectric micromotor.

43. The intravascular catheter system of claim 26, wherein the second vibration transducer is a piezoelectric micromotor.

44. The intravascular catheter system of claim 42, wherein the piezoelectric micromotor is an oscillating ceramic motor.

45. The detection and imaging system of claim 29, further comprising an imaging system having an imaging screen, wherein said processor is capable of generating images from said differentiable information.

46. A method for guiding and imaging an intravascular device through an obstruction in a blood vessel using the system of claim 28, comprising the steps of:
a) introducing said device into a blood vessel having vessel walls and an obstruction, and advancing said device until said therapeutic tip is in close proximity to said obstruction,
b) vibrating said tip at a first frequency using said first vibration transducer sufficient to advance said guide wire through said obstruction in said vessel;
c) generating a detectable signal at a second frequency using said second vibration transducer, said second frequency different than said first frequency and transmitted simultaneously therewith; and
d) generating real-time images of said therapeutic tip relative to said obstruction and said vessel walls, said real-time images generated from said detectable signals.

47. The method of claim 46, wherein said generated image facilitates the advancing of the therapeutic tip relative to the occlusion and vessel walls.

48. The method of claim 46, wherein said images are 3-dimensional images.

49. The method of claim 46, wherein said second vibration transducer is a piezoelectric micromotor, and said imaging system is an ultrasound imaging system; wherein said second vibrating step includes sending energy from said power source to said piezoelectric micromotor in a manner that generates ultrasonic frequencies.

50. The method of claim 46, wherein said catheter further comprises an angioplasty balloon, and said method further comprises the step of deploying said angioplasty balloon after said occlusion is recanalized.

51. The method of claim 46, wherein said catheter further comprises a balloon-expandable stent, and said method further comprises the step of deploying said balloon-expandable stent.

52. The method of claim 46, wherein said catheter further comprises a self-expanding stent, and the method further comprises the step of deploying said self-expanding stent.

53. The method of claim 46, wherein said images are Doppler images.

54. The method of claim 46, further comprising generating images from differentiable information using an imaging system.
